(19) United States
(12) Patent Application Publication
(10) Pub. No.: US 2005/0238619 A1
(43) Pub. Date: Oct. 27, 2005

(54) Method for the delivery of sustained release agents

(76) Inventor: Lee B. Riley, Bethlehem, PA (US)

Correspondence Address: BLANK ROME LLP, 600 NEW HAMPSHIRE AVENUE, N.W., WASHINGTON, DC, 20037, US

(21) Appl. No.: 11/082,851

(22) Filed: Mar. 18, 2005

Related U.S. Application Data
(60) Provisional application No. 60/554,230, filed on Mar. 18, 2004.

(51) Int. Cl. A61K 49/00; A61K 48/00; A61K 38/20; A61K 38/19; A61K 38/21

(52) U.S. Cl. 424/85.1; 424/85.2; 424/85.4; 514/44; 606/33

(57) ABSTRACT

The present invention relates to a method for the delivery of agents for sustained release. In particular, the present invention allows for site-specific delivery of therapeutic agents to the tissue, retention of the agents in the tissue, and sustain release of the agents from the site of delivery. The method consists of ablating the tissue and injecting an agent into the ablated tissue for sustained release.

This application claims the priority of U.S. Provisional Patent Application No. 60/554,230, filed Mar. 18, 2004.

### FIELD OF THE INVENTION

The present invention relates to a method for the delivery of agents for sustained release. In particular, the present invention allows for site-specific delivery of therapeutic agents to the tissue, retention of the agents in the tissue, and sustain release of the agents from the site of delivery.

### BACKGROUND OF THE INVENTION

With advances in antibiotics and vaccines, there has been a reduction in the seriousness of many infectious diseases; however, cancer still remains as a mostly incurable threat. One obstacle in the treatment of cancer is that the mechanisms of cancer development and propagation are diverse and poorly understood. Therefore, investigation into possible cancer treatments requires knowledge from a variety of different disciplines. Additionally, cancer patients must withstand the debilitating mental and physical effects throughout the long duration of the disease which also results in an economic burden to both the patient and the community.

Most cancers that are cured are done so by surgical resection of the primary tumor. Treatment of metastatic cancer is usually accomplished with systemic chemotherapy, though the curative results from this approach have, for the most part, been disappointing. The toxic side effects of systemic chemotherapeutic agents may be the limiting factor in determining the drug concentration delivered to the patient. In many cases these side effects preclude sufficient doses of chemotherapy agents and the tumor cells regrowth and spread.

When metastatic disease is confined to one organ (e.g. the liver), surgery or focal treatments become important. Non-surgical treatment for tumors confined to the liver include various routes of chemotherapy in which chemotherapeutic drugs are delivered directly to the liver tumors through their blood supply. As an example, the chemotherapeutic drug, fluorodeoxyuridine (FUDR), works by having a greater toxic effect on actively dividing cells such as cancer cells, rather than most normal tissues. When delivered through the liver artery, the tumors preferentially get higher concentrations of the drug relative to other cells in the body: this minimizes the overall side effects for the patient. With conventional systemic or regional treatment, the excess drug which does not contact tumor tissue degrades the condition of the healthy tissue and, therefore, can become the limiting factor in dose concentration. An ideal situation would occur if the toxic effects of the drugs could be completely localized within the liver tumor tissue without affecting the surrounding healthy tissue, enabling a higher drug concentration to completely kill all of the cancer cells.

Other tumor treatments involve starving the tumor by cutting off the blood supply to the diseased tissue. Arterial embolization has recently been recognized as an effective method for treating some liver cancers and breast cancers. According to this method, one end of a catheter is inserted into a nutrient artery leading to the cancer or tumoral tissue and an embolic agent, such as steel coils, polyvinyl alcohol sponge (IVALON™), collagen, gelatin sponge (GELFOAM™), albumin, and starch materials, is injected through the other end of the catheter to stop the blood flow. This causes necroses of the cancer or tumor tissue because it is starved of additional nutrient. One disadvantage of the transcatheter arterial embolization technique is that it is supplemented with chemotherapy. A two-step method has been tried where the artery feeding the tumor is first injected with a chemotherapy agent and then the artery is embolized. This method is usually ineffective, because the applied therapeutic agent is released from the targeted disease tissue in an extremely short time. The quick loss of the therapeutic agent is due to collateral vasculature of the cancer or tumor tissue, which tends to carry the therapeutic agent away from the diseased tissue. Recently investigators have impregnated embolic substrates (e.g. GELFOAM™) with chemotherapy agents in an effort to perfuse the tissue following the embolization process. After embolization, administering an anti-tumor agent orally or by injection is futile, because occlusion of the nutrient artery prevents the agent from reaching the cancer or tumoral tissue.

Sustained release drugs have also been developed to be delivered directly to tumor tissue. U.S. Pat. No. 6,602,524 to Batich et al. discloses encapsulation anti-cancer drugs in pH-sensitive microspheres which have a swelling transition with the pH range found in or near tumor tissue. When the microspheres swell, the loaded drug is released into the microenvironment of the tumor tissue.

U.S. Reissue Pat. No. 33,375 to Luck et al. discloses the dispersion of cytotoxic drug in a matrix, such as collagen, fibrinogen, or derivatives thereof. The matrix is then dispersed in a minor amount of a physiologically acceptable aqueous medium; and the resulting amorphous mass is injected into a lesion, e.g., tumor. Once injected, the matrix adheres to the tissue and does not migrate significantly. After injection, the drug is released into the immediate environment, so as to prevent substantial transportation of the drug to other sites, where its cytotoxic effect is undesirable. Thus, the circulating blood level of the drug remains low.

U.S. Pat. No. 4,536,387 to Sakamoto et al. discloses an anti-cancer device having an anti-cancer drug and a blood coagulation factor fixed to a structure made up of synthetic polymers such as silicone, polyesters, polyamides, polyurethanes, polyacrylonitrile, polyacrylamide, polyacrylic acid esters, polyethylene, polypropylene, polyvinyl chloride and polyvinyl alcohol; cellulosic materials and their derivatives such as cotton, hemp, pulp, ethyl cellulose and cellulose acetate; regenerated cellulose such as viscose rayon and caprammonium rayon; and various bioabsorbable materials. This anti-cancer device is used in transcatheter arterial embolization and needle therapy and slowly releases the anti-cancer drug over an extended period by residing in the cancer tissue and its nearby area.

Overall, the sustained released drugs require a modification and approval which is expensive and complicated and adds to the cost of the disease treatment.

Therefore, there remains a need for a simple and inexpensive therapeutic agent delivery system for treatment of diseased tissues, especially cancer and tumor tissues, that releases the therapeutic agent in high concentrations only within the diseased tissue while healthy tissue remains relatively unaffected. There is also a need to provide a method of implanting a substance into a tissue for sustained release into the body.

### SUMMARY OF THE INVENTION

The present inventor has discovered that ablated tissue is able to retain substances injected directly into the ablated tissue and to release the substances over a prolonged period of time.

An object of the present invention relates to a method for the treatment of diseased tissue, especially cancer or tumor tissues. In particular, the present invention allows for site-specific delivery of therapeutic agents to the diseased tissue and the sustained release of therapeutic agents from the site of delivery. The diseased tissue is first ablated, e.g. with radiofrequency (RF), microwave, ultrasound, laser, other electromagnetic radiation or heat source to kill the diseased tissue through resistive or other heating mechanisms. Because ablation often fails to kill all of the diseased cells resulting in recurrence, especially in the case of cancer or tumor, at least one therapeutic agent is then injected directly into the ablated tissue. Using this method, the therapeutic agent is retained in the tissue and released slowly overtime. The advantages of this method are many fold. First, the retention of the therapeutic agent in the tissue allows for longer contact of the diseased tissue with the therapeutic agent at higher concentration. Second, because the therapeutic agent, especially chemotherapeutic agent, can be cytotoxic, the slow release does not adversely affect healthy tissues elsewhere in the patient.

Another object of the present invention relates to a method of implanting an agent for sustained release, e.g. in an animal. A selected tissue is first ablated, e.g. with radiofrequency (RF), microwave, ultrasound, laser, or other electromagnetic radiation, to kill the tissue through heating. An agent is then injected into the ablated tissue to be released over a prolonged period of time from the injection site into the circulatory system.

Another object of the present invention relates to a method of immunotherapy, especially for cancers or tumors. The method involves first ablating the tissue to be immunized against, e.g. with radiofrequency (RF), microwave, ultrasound, laser, or other electromagnetic radiation, to kill the tissue through heating. An immunomodulatory agent (or combination of agents), such as a cytokine, is then injected into the ablated tissue to stimulate an immune response against the tissue. Because the immunomodulatory agent is retained in the ablated tissue, an immune response will develop against the specific tissue. This method is most useful in immunotherapy against cancers and tumors; however, immunotherapy against other tissues is also contemplated.

### BRIEF DESCRIPTION OF THE DRAWINGS

FIG. 1 shows photographs of fluorescein retention in ablated bovine liver over time;

FIG. 2 shows a graph of the dependence of amount fluorescein retained on the number of injections;

FIG. 3 shows a graph depicting the exponential release of fluorescein from ablated bovine liver tissue;

FIG. 4 shows a graph depicting the residual fluorescein after 1, 3, and 8 days in mouse tumors treated with RFA and injected with fluorescein. The left images are taken under visible light, while the right images are corresponding images under Wood's lamp;

FIG. 5 shows a graph depicting fluorecein concentration in urine of six patients whose tumors were treated with RFA and injected with fluorescein; and

FIG. 6 shows a graph depicting fluorecein concentration in urine of one of the six patients in FIG. 5 and the calculated fluorescein retained in the tumor.

### DETAILED DESCRIPTION OF THE PREFERRED EMBODIMENTS

As used in the present invention, tissue ablation can be performed by any method known in the art, such as with radiofrequency (RF), microwave, ultrasound, laser, or other electromagnetic radiation, to kill the diseased tissue through resistive or other mechanisms of heating. The preferred method is radiofrequency ablation (RFA). In RFA, an electrode is inserted into the diseased tissue, such as cancer or tumor tissues, and current passing from the electrode into the patient (to an electrical return typically being a large area plate on the patient's skin) destroys the diseased tissue through resistive heating. A simple RFA electrode is a conductive needle having an uninsulated tip placed within the diseased tissue. The needle is energized with respect to a large area contact plate on the patient's skin by an oscillating electrical signal of approximately 460 kHz. Current flowing radially from the tip of the needle produces a spherical or ellipsoidal zone of heating (depending on the length and shape of the exposed needle tip(s)) and ultimately a lesion within a portion of the zone having sufficient temperature to kill the cells within the zone. The electrode energy is limited to avoid charring, boiling and vaporization of the tissue next to the electrode, a condition that greatly increases the resistance between the electrode and the remainder of the diseased tissue. The tissue next to the electrode chars first because of the high current densities close to the electrode and thus creates a bottleneck in energy transfer.

Several improvements and techniques have been developed to enhance RFA, all of which are appropriate for the present invention. Apparatuses and methods for RFA can include, but are not limited, to those disclosed in U.S. Pat. No. 6,280,441 to Ryan and U.S. Pat. No. 6,663,622 to Foley et al.; and U.S. Patent Application Publication Nos. 2004/0133196 to Schaefer et al., 2004/0236322 to Mulier et al., 2005/0010209 to Lee, Jr. et al., and 2002/0022864 to Nahvi et al.; the disclosures of which are incorporated herein by reference.

Once the diseased tissue is ablated, at least one agent in injected into the ablated tissue. For a given volume of an agent, multiple injections are more preferable than a single injection. For example, if 1 mL of an agent are to be injected into the ablated tissue, it is preferred that ten injections of 0.1 mL each are effected, rather than a single injection of 1 mL. Other devices can be developed to disperse the agent in the ablated tissue. For example, a injection device containing more than one needles for simultaneous delivery of the agent to multiple sites within the ablated tissue can be used to practice the present invention.

The therapeutic agent can be, but is not limited to, small molecules, proteins, peptides, nucleic acids (DNA or RNA), cells, drugs, or combinations thereof. Illustrative agents include chlorambucil, melphalan, busulfan, carmustine, lomustine, streptozotocin, thiotepa, decarbazine methotrexate, 5-fluorouracil, cytarabine, azaribine mercaptopurine, thioguanine, vinblastine, vincristine, actinomycin D, adriamycin, bleomycin, mithramycin, mitomycin C, L-asparaginase, cisplatin, procarbazine, prednisone, prednisilone, triamicinolone, testosterone, estrogen, insulins, hydroxyurea, immuno-modulatory agents, antibodies, antibiotics, antivirals, and combinations thereof. The immuno-modulatory agents can be, but are not limited to, cytokines, such as interleukins, interferons, growth factors, chemokines (agents that attract cells into an area), tumor antigens, modified tumor antigens, DNA/RNA that modify the immune system, or cells, such as those from the immune system. Additionally agents that would be used to further delay the release of any of these agents could be used. The therapeutic agents may be used individually or in combination, depending upon the nature of the agent, the disease, and whether cooperative action is pharmacologically indicated. The agent can be further modified, by modifying the drug, particularly by bonds which allow for enzymatic cleavage, e.g., hydrolysis, or by introducing materials into the composition which will aid in the maintenance of the retention of the drug at the site of introduction; however, it is preferred that the agent is not modified because the inventor has discovered that the ablated tissue, possibly due to its lack of vasculature, is sufficient by itself in retaining the agent for an extended period of time with a slow rate release from the site of delivery.

Without further description, it is believed that one of ordinary skill in the art can, using the preceding description and the following illustrative examples, make and utilize the compounds of the present invention and practice the claimed methods. The following example is given to illustrate the present invention. It should be understood that the invention is not to be limited to the specific conditions or details described in this example.

### EXAMPLE 1

### Fluorescein Retention in RF Ablated Tissue

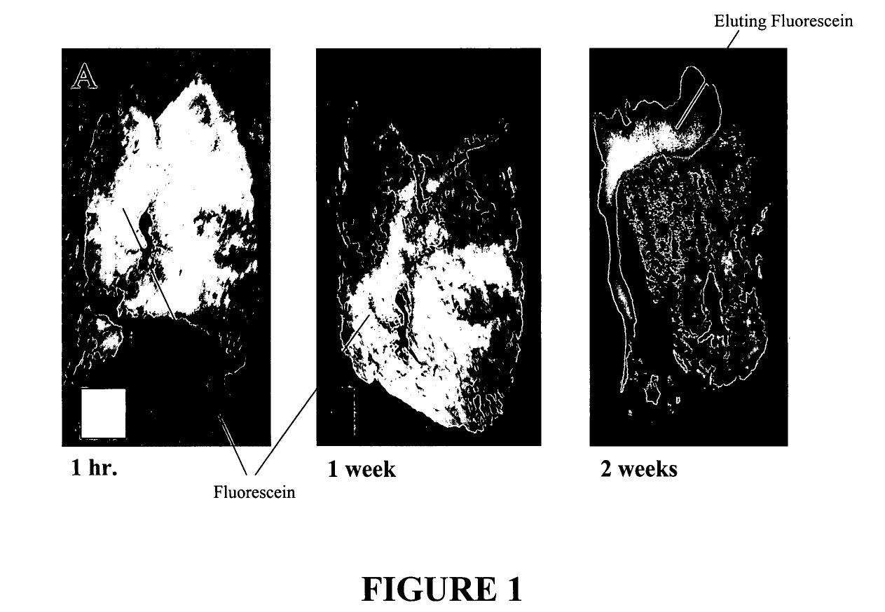

Bovine liver was ablated, injected multiple times with fluorescein. To demonstrate the effectiveness of the injections, the tissue was bivalved and agitated in 1 L of phosphate buffered saline (PBS) for extended periods of time. The PBS was exchanged daily for two weeks. At weekly intervals, the ablated tissue was photographed under ultraviolet (UV) light. According to FIG. 1, fluorescein was easily detected within the ablated tissue for at least two weeks, at which point, fluorescein was still being eluted from the ablated tissue (Panel C of FIG. 1). This approach can be used to help train and assess people to use the technique.

### EXAMPLE 2

### Multiple Injections Improved Fluorescein Retention by RF Ablated Tissue

Ablated bovine liver, prepared as in Example 1, was injected 3, 10, or 30 times with fluorescein (total volume of 0.5 mL). The tissue was extensively washed over 10 minutes; and the retained fluorescein was calculated as a percentage of the total injected fluorescein. To quantify the retained fluorescein, the washed tissue was homogenized, centrifuged and the supernatant assessed for fluorescein using a fluorimeter with excitation and emission filters of 485 nm and 525 nm, respectively.

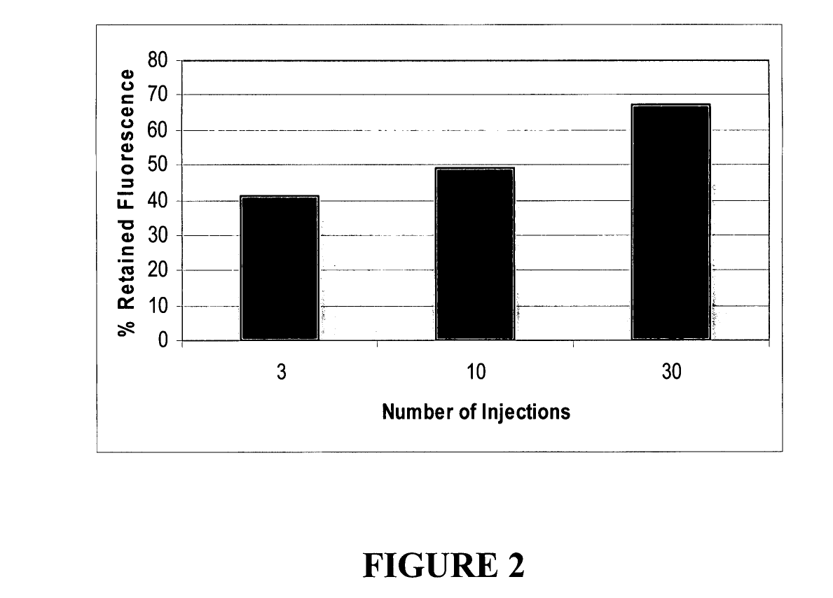

According to FIG. 2, up to about 65% of the injected fluorescein was retained by the RF ablated liver when 30 injections were used, while the 10 and 3 injections retained 49% and 41% of the fluorescein, respectively. This clearly indicated that the amount of fluorescein retention correlated positively with the number of injections when using a single needle device. Multi-needle devices could be developed to accomplish similar results.

### EXAMPLE 3

### Exponential Release of Fluorescein from RF Ablated Tissue

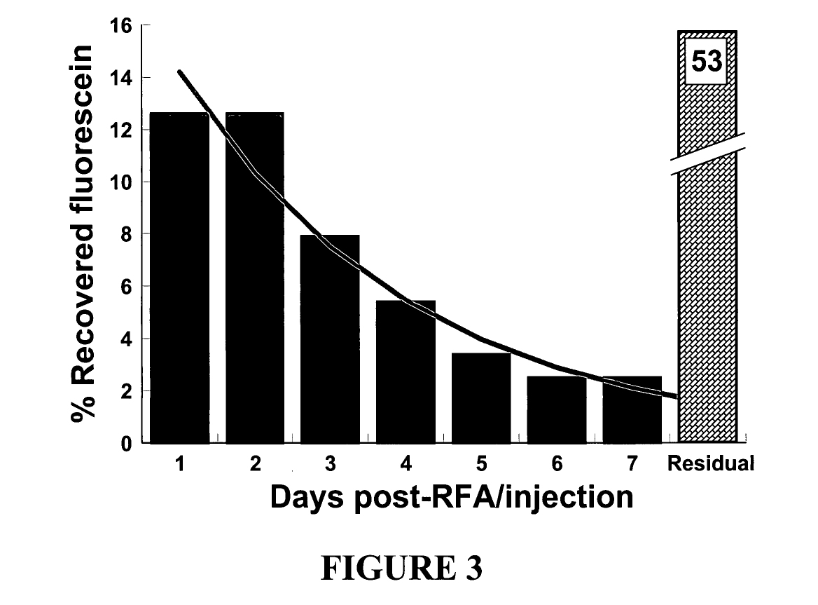

To determine the rate of fluorescein release, bovine liver was ablated with RF and injected with fluorescein. According to FIG. 3, which depicted the amount of fluorescein released by the ablated tissue as well as the retained fluorescein as a percentage of the total recovered fluorescence, the released fluorescein followed an exponential decline. However, after one week of continued washing, the ablated tissue still contained greater than 50% of the original fluorescein. These data demonstrated in an ex vivo model that RF ablated tissue provides a mechanism to deliver high local concentrations of molecules that can be slowly released over prolonged periods of time.

### EXAMPLE 4

### RF Ablated, Subcutaneous Tumors Retain Fluorescein in an In Vivo Model

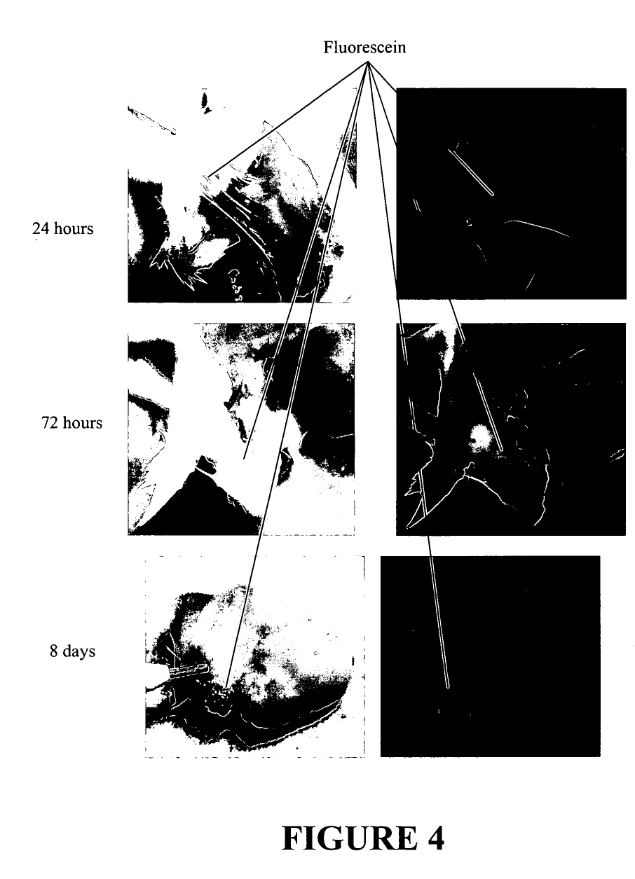

Balb/c female mice were injected with 10⁶ Ras-6 tumor cells in the flank. The Ras-6 tumor was derived by transfection of a p53-null BALB/c murine embryo fibroblast line with a mutant ras gene. Once the tumors reached 0.5-1 cm in size, the animals were placed under general anesthesia; and the tumors was ablated using a 0.7 cm multi-tined probe. RFA was accomplished by starting with 1-3 watts of power and increasing the power every 30 seconds. A special resistor circuit was placed in the circuit to avoid a low resistance default interrupt error built into the RFA generator. Following RFA, the tumors were injected with a total volume of 50 μl of fluorescein using 1 to 3 injections. At various intervals, the animals were sacrificed and the retained fluorescein assessed. FIG. 4 depicts the residual fluorescein after 1, 3, and 8 days. Consistent with the ex vivo studies, the amount of fluorescein decreases over this time interval; however, substantial fluorescein remains in the ablated tumor for ar least 8 days in vivo.

### EXAMPLE 5

### RF Ablated, Hepatic Tumors Retain Fluorescein in Clinical Studies

Six patients with unresectable hepatic metastases or hapatocellular carcinoma were enrolled in the Internal Review Board approved protocol following the process of informed consent. Eligibility required that the patient receive RFA for therapeutic intent. RFA was performed using a RadioTherapeutics® device according to their specifications. Subsequently, 1 ml of 10% fluorescein was injected into the ablated tissue (20 different injections).

Because 98% of fluorescein is excreted in the urine, the amount of released and retained fluorescein could be estimated from serial urine sample. Serially, first morning void urine samples were collected and assessed for fluorescein. The retained fluorescein was calculated by subtracting the estimated excreted fluorescein (measured fluorescein (mg/mL)×estimated 2 L urine/day volume) from the total injected fluorescein (100 mg).

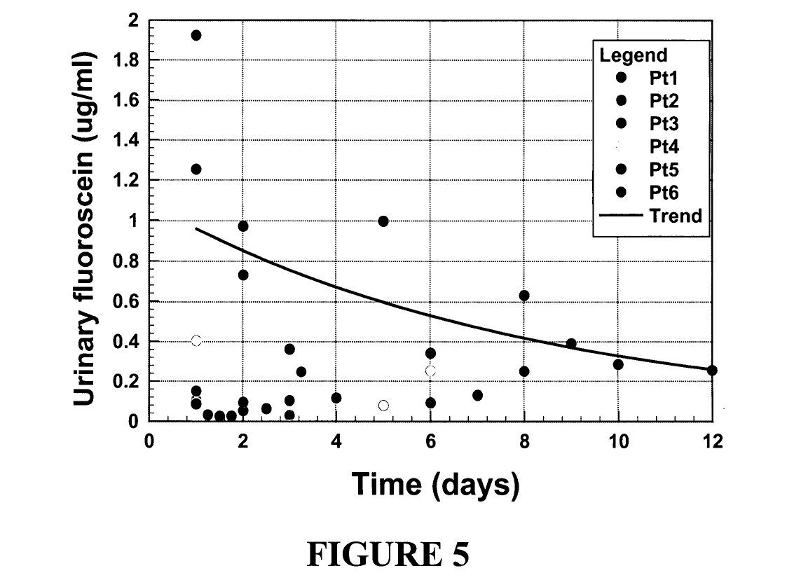

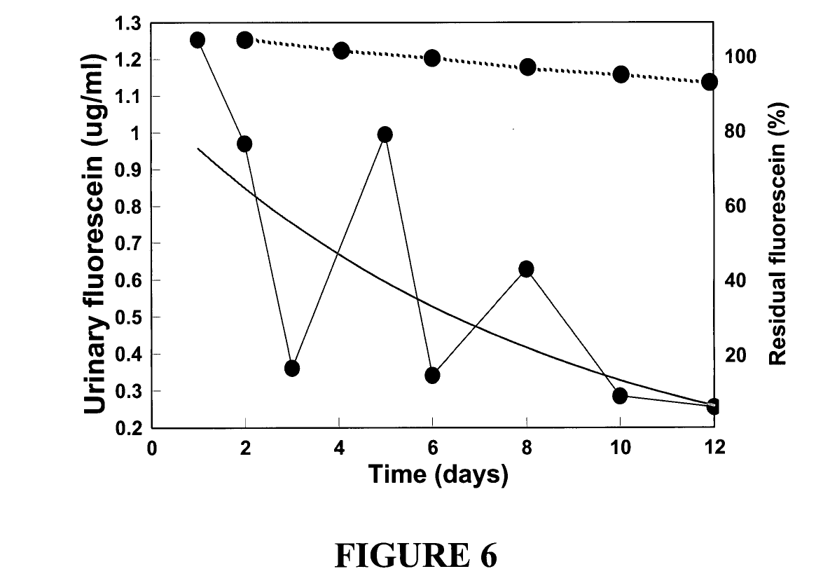

FIG. 5 shows the fluorescein concentration in urine for the six patients over 12 days. The black line represents the trend for all six patients. FIG. 6 depicts the data for patient 1. The dotted line represents the calculated fluorescein retained in the ablated tumor after 12 days, which showed a retention of greater than 90% after 12 days.

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

1. A method for treating a tumor tissue comprising the steps of
a) ablating the tumor tissue; and
b) injecting the ablated tumor tissue with a therapeutic agent.

2. The method of claim 1, wherein the therapeutic agent is a chemotherapeutic agent.

3. The method of claim 1, wherein the therapeutic agent is a immuno-modulatory agent.

4. The method of claim 1, wherein the therapeutic agent is selected from the group consisting of cytokine, interferon, growth factor, chemokines, tumor antigens, modified tumor antigens, DNA/RNA, or cells.

5. The method of claim 1, wherein step b) contains multiple injections.

6. The method of claim 1, wherein step a) comprises radiofrequency ablation (RFA).

7. A method for treating a diseased tissue comprising the steps of
a) ablating the diseased tissue; and
b) injecting the RF ablated diseased tissue with an agent for treating said disease.

8. The method of claim 7, wherein the agent is selected from the group consisting of antibiotics, antivirals, antibodies, cytokines, chemokines, growth factors, immunomodulatory compounds and combinations thereof.

9. The method of claim 7, wherein step b) contains multiple injections.

10. The method of claim 7, wherein step a) comprises radiofrequency ablation (RFA).

11. A method for implanting a composition for sustained release comprising the steps of
a) ablating a selected tissue; and
b) injecting the ablated diseased tissue with the drug.

12. The method of claim 11, wherein the selected tissue is normal tissue.

13. The method of claim 11, wherein the selected tissue is muscle tissue or liver tissue.

14. The method of claim 11, wherein the composition is selected from the group consisting of drugs, hormones, peptides, proteins, nucleic acids, tumor antigens, chemokines, and combinations thereof.

15. The method of claim 10, wherein step b) contains multiple injections.

16. The method of claim 10, wherein step a) comprises radiofrequency ablation (RFA).
